(19) United States
(12) Patent Application Publication
(10) Pub. No.: US 2019/0070413 A1
(43) Pub. Date: Mar. 7, 2019

(54) VESTIBULAR PROSTHESES AND ASSOCIATED SYSTEMS AND METHODS

(71) Applicants: James O. Phillips, Seattle, WA (US); Steven M. Bierer, Seattle, WA (US); Leo Ling, Seattle, WA (US); Kaibao Nie, Bothell, WA (US); Jay Rubinstein, Seattle, WA (US)

(72) Inventors: James O. Phillips, Seattle, WA (US); Steven M. Bierer, Seattle, WA (US); Leo Ling, Seattle, WA (US); Kaibao Nie, Bothell, WA (US); Jay Rubinstein, Seattle, WA (US)

(73) Assignee: University of Washington, Seattle, WA (US)

(21) Appl. No.: 13/526,250

(22) Filed: Jun. 18, 2012

Related U.S. Application Data
(60) Provisional application No. 61/498,117, filed on Jun. 17, 2011.

(51) Int. Cl. A61N 1/36; A61B 5/00; A61N 1/375; A61N 1/372

(52) U.S. Cl. CPC A61N 1/36038; A61B 5/6867; A61B 5/11; A61N 1/37211; A61N 1/375

(57) ABSTRACT

The present technology is directed to vestibular prostheses and associated systems and methods. In several embodiments, for example, a vestibular prosthesis includes an external rotational sensor configured to receive velocity and orientation information. The prosthesis further includes an external processor configured to convert the velocity and orientation information into an audio signal. The audio signal is sent to a cochlear implant, which applies stimulation to a semicircular ear canal in response to receiving the audio signal. In some embodiments, the processor and cochlear implant communicate in real time via an inductive link.

### CROSS-REFERENCE TO RELATED APPLICATION(S)

This application claims the benefit of U.S. Provisional Patent Application No. 61/498,117, filed Jun. 17, 2011, which is incorporated herein by reference in its entirety.

### STATEMENT REGARDING FEDERALLY SPONSORED RESEARCH

This invention was made with government support under HHS-N-260-2006-00005-C awarded by National Institutes of Health (NIH). The government has certain rights in the invention.

### TECHNICAL FIELD

The present technology is directed toward vestibular prostheses and associated systems and methods.

### BACKGROUND

Vestibular disorders can be disabling conditions which result in imbalance, disorientation, and oscillopsia. There are currently no effective clinical strategies for the restoration of vestibular function following the loss of hair cells in the vestibular labyrinth. One strategy for treating vestibular loss is to replace natural vestibular sensation with electrical stimulation of the afferent vestibular nerve fibers using an implanted stimulator. The electrical stimulus can restore the afferent signal carried by the vestibular nerve, producing a restoration of movement-elicited behavior. A partial loss of vestibular function can also be treated with this strategy if the prosthesis allows natural sensation of rotation by the remaining intact hair cells.

Such a treatment requires that electrical stimulation be combined with natural stimulation to produce a summed response. However, the current designs of vestibular prostheses have many limitations. Most devices are implanted in the ampullae of individual canals, which can potentially compromise natural vestibular sensitivity of the implanted ear. The devices are also constructed to be totally implantable, which poses challenges in terms of power consumption and reliability, and limits the upgradability of the device. Accordingly, there exists a need for improved treatments for vestibular disorders.

### BRIEF DESCRIPTION OF THE DRAWINGS

FIG. 1 is a schematic illustration of a vestibular prosthesis configured in accordance with embodiments of the present technology.

FIG. 2 is an isometric view of a cochlear implant configured for use with a vestibular prosthesis in accordance with embodiments of the present technology.

FIG. 3 is a partially schematic, isometric view of a vestibular prosthesis casing configured in accordance with embodiments of the present technology.

FIG. 4 is a partially schematic, exploded isometric view of a bone anchor attachment for a vestibular prosthesis configured in accordance with embodiments of the present technology.

### DETAILED DESCRIPTION

The present technology is directed to vestibular prostheses and associated systems and methods. In several embodiments, for example, a vestibular prosthesis includes an external rotational sensor configured to receive velocity and orientation information. The vestibular prosthesis further includes an external processor configured to convert the velocity and orientation information into an audio signal. The audio signal is provided to a cochlear prosthesis, which applies stimulation to a semicircular ear canal in response to receiving the audio signal. In some embodiments, the processor and cochlear prosthesis communicate in real time via an inductive link of the cochlear prosthesis.

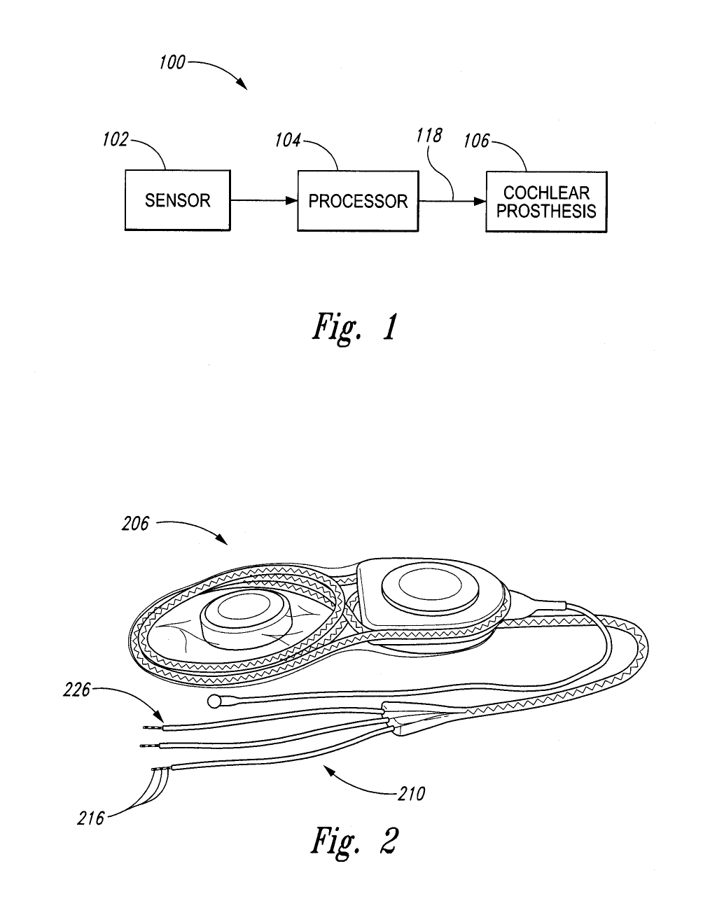

Certain specific details are set forth in the following description and in FIGS. 1-4 to provide a thorough understanding of various embodiments of the technology. Other details describing well-known structures and systems often associated with vestibular prostheses, cochlear implants, and other associated devices have not been set forth in the following disclosure to avoid unnecessarily obscuring the description of the various embodiments of the technology. A person of ordinary skill in the art, therefore, will accordingly understand that the technology may have other embodiments with additional elements, or the technology may have other embodiments without several of the features shown and described below with reference to FIGS. 1-4.

FIG. 1 is a schematic illustration of a vestibular prosthesis 100 configured in accordance with embodiments of the present technology. The vestibular prosthesis 100 includes a sensor 102 configured to receive information related to position, velocity (e.g., head rotational velocity), acceleration, and/or orientation. In some embodiments, the sensor 102 can receive this information in the form of a voltage signal provided by a rotational sensor, accelerometer, gyroscope, rate sensor, or other device to sense a patient's roll, pitch and yaw. The sensor 102 can be coupled to an external location on a person (e.g., to a person's head).

The vestibular prosthesis 100 further includes a processor 104 configured to transform the signal received by the sensor 102. In several embodiments, for example, the processor 104 converts the velocity signal from the sensor 102 into an audio signal. In some embodiments, the output of the processor 104 can be a combination of multiple carriers. For example, the output could comprise the output of a right anterior canal channel, right posterior canal channel, and right lateral canal channel, each of which can be amplitude modulated by head velocity. In some embodiments, one or more of these channels can be scaled, amplified, and/or filtered. As will be described in further detail below, the transformation and combination of these carriers can provide for tailored stimulation treatments. While FIG. 1 illustrates the sensor 102 and processor as separate components, in some embodiments the vestibular prosthesis 100 comprises a joint external sensor/processor.

The vestibular prosthesis 100 further includes a cochlear implant 106 configured to receive the transformed signal and/or commands from the processor 104 and apply stimulation to the inner ear in response to receiving the transformed signal. The cochlear implant 106 can be any cochlear implant known in the art. In a particular embodiment, the signal receiver/stimulator is a Nucleus Freedom cochlear implant made by Cochlear Limited.

As will be discussed in further detail below with reference to FIG. 2, the cochlear implant 106 can include several features generally similar to traditional cochlear implant devices. For example, the cochlear implant 106 can include an external processor and an internally-implantable device having stimulation leads. The sensor 102 and/or processor 104 can externally communicate with the external portion of the cochlear implant 106. The external portion of the cochlear implant 106 can then relay energy, information, data, and/or commands to the implanted portion. The implanted stimulation leads can be placed in a person's inner ear (e.g., in a perilymphatic space adjacent to an ampulla of a semicircular canal) and apply stimulation energy within the semicircular canal to preserve rotational sensitivity in the implanted ear.

The stimulation parameters can be controlled by pre-set programming or can be modified by a user or practitioner. For example, the cochlear implant 106 can be programmed to produce patterned electrical stimuli that do not require a head-related input signal. In some embodiments, a graphical user interface can control parameters such as pulse width (phase) and amplitude. Active and return electrodes can be configured for a monopolar or bipolar modes of stimulation. By manipulating a duration parameter, a single pulse or train of pulses can be chosen. In addition, a pulse train can be sinusoidally amplitude-modulated or frequency-modulated based on specified modulation limits and oscillation rate. Combined electrical and rotational stimulation can result in a summation of responses, thereby providing improved vestibular function.

The rate of stimulation by the leads 100 can vary to achieve the desired vestibular response. In some embodiments, for example, it may be physiologically desirable to be able to decrease the firing rate of a spontaneously active population of neurons. High-rate stimulation can be used to induce a partial depolarization block so that the firing rate can be reduced below what it would be at if there was no electrical stimulation. This allows the vestibular prosthesis 100 to both increase and decrease the spontaneous firing rate of its target neurons.

The sensor 102, processor 104, and/or cochlear implant 106 can communicate by wired or wireless connections, or a combination thereof. In some embodiments, for example, the processor 104 and cochlear implant 106 can be connected by an inductive link 118 (e.g., a radiofrequency link). The inductive link 118 can include an inductive power transfer unit. The inductive link 118 can provide the cochlear implant 106 with the required power from a wireless power supply system (e.g., a power supply system powering to the processor 104) to enable long-term cochlear implant operation without a large internal power source. In further embodiments, the cochlear implant itself 106 contains the inductive link 118. For example, the external portion of the cochlear implant 106 can communicate with the internally-implanted portion via the inductive link 118.

In some embodiments, the sensor 102, processor 104, and/or cochlear implant 106 can communicate in real-time. One or both of the sensor 102 and processor 104 can be integrated with the cochlear implant 106 or can comprise a separate component. In several embodiments, for example, the sensor 102 and processor 104 can be externally positioned while at least a portion of the cochlear implant 106 is internally positioned. As will be discussed in further detail below with reference to FIG. 4, all or a portion of the vestibular prosthesis can be configured for temporary, removable attachment to a person or permanent attachment.

FIG. 2 is an isometric view of a cochlear implant 206 configured for use with a vestibular prosthesis in accordance with embodiments of the present technology. The cochlear implant 206 can be generally similar to the cochlear implant 106 described above with reference to FIG. 1. For example, the cochlear implant 106 can be a receiver stimulator configured to receive a signal (e.g., an audio signal) and apply stimulation to an inner ear based on this signal. The cochlear implant 206 includes a plurality of stimulation, leads 210 configured to apply the stimulation energy. In further embodiments, the cochlear implant 206 can include other features of cochlear implants known in the art.

In the illustrated embodiment, the leads 210 have a thin (approximately 140 μm diameter) distal portion 226 that is about 2.5 mm in length, which is designed to be inserted in the perilymphatic space adjacent to the ampulla of each semicircular canal. The leads 210 can have other dimensions in other embodiments. In some embodiments, the stimulation leads 210 comprise an electrode array. Each lead 210 can contain multiple (e.g., three) independent stimulation sites 216 that are 200-250 μm in length. The inserted distal portions 226 are designed to not occlude the lumen of the membranous labyrinth, and they can be implanted without impinging on the crista ampularis, thereby preserving the rotational sensitivity of the implanted canal.

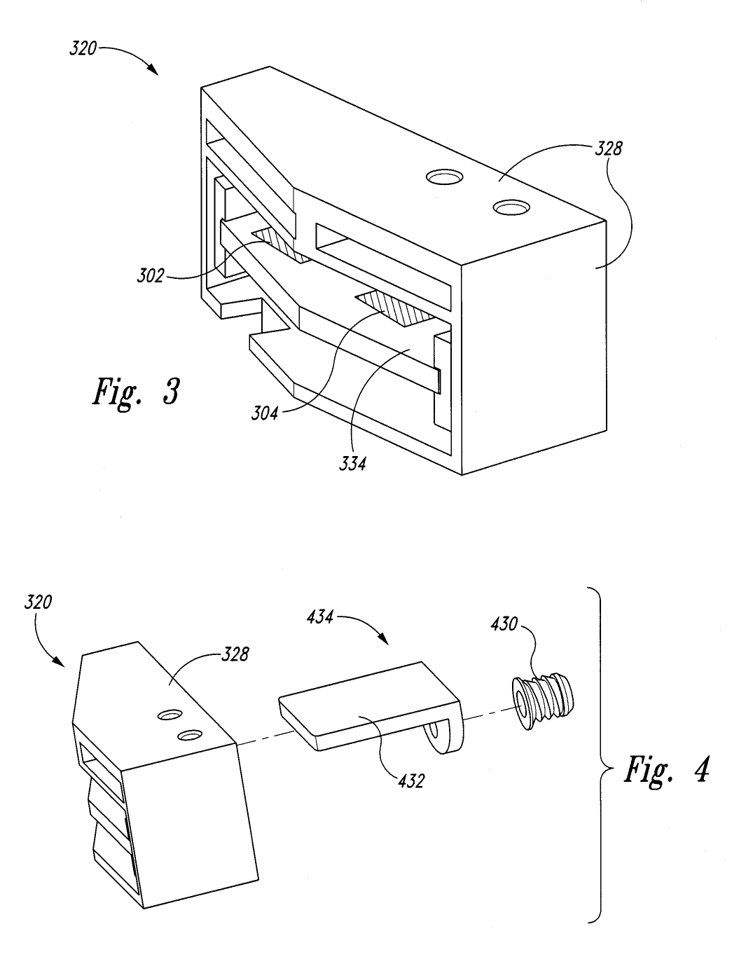

FIG. 3 is a partially schematic, exposed isometric view of a vestibular prosthesis casing 320 configured in accordance with embodiments of the present technology. The casing 320 comprises a plurality of sidewalls 328 configured to at least partially enclose a sensor 302 and a processor 304 generally similar to the sensor 102 and processor 104 described above with reference to FIG. 1. In some embodiments, the sensor 302 and processor 304 are mounted on the same or multiple circuit boards 334. In further embodiments, the casing 320 can house multiple sensors 302 and/or processors 304.

The sensor 302 and processor 304 can interface with a cochlear implant (e.g., the cochlear implant 206 described above with reference to FIG. 2) by means of an inductive link or other form of electronic communication. While the internal portion of the casing 320 is illustrated for purposes of discussion, the sensor 302 and processor 304 can be completely enclosed in the casing 320 in further embodiments with the inclusion of additional sidewalls 328. The casing 320 can comprise plastic, metal, or other materials or combinations of materials.

FIG. 4 is a partially schematic, exploded isometric view of a bone anchor attachment 434 for use with a vestibular prosthesis configured in accordance with embodiments of the present technology. The bone anchor attachment 434 is illustrated in use with the vestibular prosthesis casing 320 described above with reference to FIG. 3. The bone anchor attachment 434 includes a screw 430 coupled to the vestibular prosthesis casing 320 with an orientation tab 432. The orientation tab 432 can attach to a sidewall 328 of the vestibular casing 320 with screws, fasteners, adhesive, or other attachment mechanism. The screw 430 can be a biocompatible osseointegrated percutaneous screw configured for attaching the orientation tab 432 to a skull or other bone. The bone anchor attachment 434 allows for a sensor and processor (e.g., sensor 302 and processor 304) to be externally attached to a person's head.

The bone anchor attachment 434 can provide permanent or releasable attachment between the casing 320 and the skull. For example, in some embodiments, the bone anchor attachment 434 can further comprise a release mechanism (e.g., a quick-release pin) to readily detach the casing 320 from the skull. By fixedly attaching the casing 320 to the skull, the bone anchor attachment 434 can provide reliable transmission of very small accelerations and displacements over a full range of frequencies to the external sensors as required for a successful vestibular prosthesis.

The present technology offers several advantages over fully implantable systems. First, by having an external sensor and processor, these components can be serviced, replaced, or upgraded without additional surgeries, extending the usable life of the implanted prosthesis by allowing the overall device to incorporate technological advances through replacement of the external components only. Further, the implantable portion of the device, the cochlear prosthesis, can use existing, highly reliable receiver-stimulator technology. Further, by using an external processor coupled to the cochlear implant by inductive link, power consumption demands can be reduced and the reliability of the design can be increased.

From the foregoing, it will be appreciated that specific embodiments of the technology have been described herein for purposes of illustration, but that various modifications may be made without deviating from the disclosure. Certain aspects of the new technology described in the context of particular embodiments may be combined or eliminated in other embodiments. Additionally, while advantages associated with certain embodiments of the new technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A vestibular prosthesis, comprising:
a receiver configured to receive a velocity voltage signal;
an external processor configured to convert the velocity voltage signal to an audio signal; and
a cochlear implant configured to receive the audio signal and apply stimulation in response to receiving the audio signal.

2. The vestibular prosthesis of claim 1, further comprising an external rotational sensor configured to sense at least one of position, orientation, velocity, and acceleration.

3. The vestibular prosthesis of claim 1 wherein the cochlear implant further comprises a stimulation lead configured for placement in a perilymphatic space adjacent to an ampulla of a semicircular canal and further configured to apply stimulation energy within the semicircular canal.

4. The vestibular prosthesis of claim 3 wherein the stimulation lead comprises an electrode array.

5. The vestibular prosthesis of claim 1, further comprising an osseointegrated percutaneous screw configured for attaching at least a portion of the vestibular prosthesis to a skull.

6. The vestibular prosthesis of claim 1 wherein the processor is coupled to the cochlear implant with a real-time communication link.

7. A vestibular prosthesis configured for attachment to a patient, comprising:
an external sensor configured to sense motion and orientation of the patient's head;
an external processor configured to receive a signal related to the motion and orientation and perform a transformation on the signal; and
an implant configured for placement in the inner ear, wherein the implant is configured to apply stimulation according to the transformed signal.

8. The vestibular prosthesis of claim 7 wherein the prosthesis is configured for temporary, removable attachment to the patient.

9. The vestibular prosthesis of claim 7 wherein the external sensor comprises a rotational sensor.

10. The vestibular prosthesis of claim 7 wherein the transformed signal comprises an audio signal.

11. The vestibular prosthesis of claim 7 wherein the external sensor is configured to sense velocity and acceleration of the patient's head.

12. The vestibular prosthesis of claim 7 wherein the implant comprises a plurality stimulation leads.

13. The vestibular prosthesis of claim 7 wherein the processor is coupled to the implant with an inductive link.

14. An external, physical, computer-readable storage medium having stored thereon, computer-executable instructions that, if executed by a computing system, cause the computing system to perform operations comprising:
receiving at least one of position, velocity, acceleration, or orientation data from a sensor;
transforming the data; and
passing the transformed data to a cochlear implant.

15. The computer-readable storage medium of claim 14 wherein transforming the data comprises transforming the data into an audio signal.

16. The computer-readable storage medium of claim 14 wherein the operations further comprise instructing the cochlear implant to apply stimulation to a treatment site in an inner ear.

17. The computer-readable storage medium of claim 14 wherein the operations further comprise supplying power to the cochlear implant.

18. The computer-readable storage medium of claim 14 wherein passing the transformed data to the cochlear implant comprises wirelessly passing the transformed data to the cochlear implant.

19. The computer-readable storage medium of claim 14 wherein passing the transformed data to the cochlear implant comprises passing the data in real-time.

20. The computer-readable storage medium of claim 14 wherein transforming the data comprises filtering, amplifying, or scaling the data.
